(19) United States
(12) Patent Application Publication
(10) Pub. No.: US 2022/0071498 A1
(43) Pub. Date: Mar. 10, 2022

(54) PHYSIOLOGY SENSING APPARATUS, PHYSIOLOGY SENSING METHOD, AND PHYSIOLOGICAL INFORMATION SERVICE SYSTEM

(71) Applicant: MEDX TECHNOLOGY INC., Taipei City (TW)

(72) Inventors: Chia-Ming Hsu, Taipei (TW); Yi-Yuan Chen, Taipei (TW)

(73) Assignee: MEDX TECHNOLOGY INC., Taipei City (TW)

(21) Appl. No.: 17/418,656

(22) PCT Filed: Dec. 10, 2019
(86) PCT No.: PCT/CN2019/124247

§ 371 Date: Jun. 25, 2021

(30) Foreign Application Priority Data
Dec. 27, 2018 (CN) 201811606604.5

(51) Int. Cl. A61B 5/0205; A61B 5/00; A61B 5/27

(52) U.S. Cl. CPC A61B 5/02055; A61B 5/6802; A61B 5/4806; A61B 2562/0247; A61B 5/27; A61B 2562/0295; A61B 2562/0271; A61B 5/0022

(57) ABSTRACT

According to the present invention, a physiological sensing apparatus is detachably provided on a textile. The physiological sensing apparatus includes a sensing module and a processing module, and the sensing module is used for receiving a touch event to generate a physiological sensing signal. The sensing module includes a sensor element and a first film; the sensor element is used for detecting the physiological sensing signal; a first surface of the first film includes a conductive ink pattern; a first surface of the sensor element is superimposed on the first surface of the first film, so that the conductive ink pattern is in contact with the sensor element. The processing module is coupled with the sensing module and is used for receiving the physiological sensing signal to determine a physiological event corresponding to a change in physiological sensing signals.

### CROSS-REFERENCE TO RELATED APPLICATIONS

This application is an U.S. national phase entry of International Application PCT/CN2019/124247, filed on Dec. 10, 2019 which claims the priority right of Chinese Patent Application No. 201811606604.5, filed in Chinese Patent Office on Dec. 27, 2018, and entitled “PHYSIOLOGICAL SENSING APPARATUS, PHYSIOLOGICAL SENSING METHOD, AND PHYSIOLOGICAL INFORMATION SERVICE SYSTEM”, the entire contents of which are incorporated herein by reference.

### TECHNICAL FIELD

The present disclosure relates to a sensing apparatus, a sensing method, and a service system, and particularly to a sensing device, a sensing method, and a service system applied in the field of physiological information.

### BACKGROUND

Based on the technology development of wearable electronic devices, the combination of electronic devices and sensing elements to provide more diverse services closer to life has been unstoppable. States of bodily functions are concerned by most people. Methods of measuring physiological parameters include measuring with common measurement products used in hospitals, such as an electrocardiogram monitor, a blood oxygen monitor, and a thermometer.

However, the measurement products usually have certain sizes, causing trouble during measurement and even difficulty in moving these measurement products. If users want to measure the physiological parameters, they have to move to the sides of the measurement products for operation, so it is inconvenient for the users who need to examine physiological parameters every day. Thus, it is urgent to provide a means of overcoming these difficulties to enhance human life quality.

### SUMMARY

A simplified summary of this disclosure is provided such that readers may have a basic understanding of the disclosure. The summary is not a complete description of the present invention, and it is not intended to indicate important/key components of the embodiments of the present invention or define the scope of the present invention.

According to one or more embodiments of the present disclosure, a physiological sensing device is disclosed. The physiological sensing apparatus is detachably provided on a textile, wherein the physiological sensing apparatus includes a sensing module and a processing module. The sensing module is configured to receive a contact event to generate a physiological sensing signal; and the sensing module includes a sensor element and a first film. The sensor element is configured to detect the physiological sensing signal. A first surface of the first film includes a conductive ink pattern, and a first surface of the sensor element is superimposed on the first surface of the first film, so that the conductive ink pattern is in contact with the sensor element; and the processing module is coupled to the sensing module. The processing module is configured to receive the physiological sensing signal to determine a physiological event corresponding to a change in the physiological sensing signal.

In one or more embodiments, the sensing module further includes a conductive film, which is arranged to cover an electrode pattern of the conductive ink pattern such that the conductive film is between the electrode pattern and the sensor element.

In one or more embodiments, the sensor element further includes a second film, which is arranged to cover the sensor element and a circuit pattern of the conductive ink pattern.

In one or more embodiments, the conductive ink pattern further includes a comb structure.

In one or more embodiments, the comb structure includes a plurality of strip-shaped resistors arranged in parallel, and first ends of the resistors in the same direction are connected to each other such that the resistors form a parallel circuit.

In one or more embodiments, the comb structure includes a plurality of strip-shaped first resistors arranged in parallel and a plurality of strip-shaped second resistors arranged in parallel, first ends of the first resistors are connected to each other such that the first resistors form a parallel circuit, and second ends of the second resistors are connected to each other such that the second resistors form a parallel circuit, wherein the first ends are opposite to the second ends.

In one or more embodiments, the first resistors and the second resistors are arranged alternately.

In one or more embodiments, the sensing module further includes a spacer, and the spacer is arranged on a second surface of the sensor element and a first surface of the second film such that the spacer is between the sensor element and the second film, wherein the second surface of the sensor element is an opposite surface of the first surface of the sensor element.

In one or more embodiments, the sensor element is a fabric sensor.

In one or more embodiments, the fabric sensor includes a protruding fabric portion structure.

In one or more embodiments, the conductive ink pattern printed on the first surface of the first film contains a material with high electrical conductivity.

In one or more embodiments, the conductive ink pattern includes an electrode pattern and a circuit pattern.

In one or more embodiments, the sensor element includes a semiconductor sensor or a semiconductor sensing module, wherein the semiconductor sensor includes at least one of a temperature sensor and a pressure sensor, and the semiconductor sensing module includes at least one of a Bluetooth module and a wireless transmission module.

In one or more embodiments, the sensing module includes one or more of the sensor elements.

In one or more embodiments, the first film includes an elastic waterproof film.

In one or more embodiments, the sensing module includes a conductive film printed with an elastic conductive ink.

In one or more embodiments, the physiological sensing apparatus further includes a waterproof film, and the waterproof film is arranged above the sensor element and the first film such that the sensor element and the first film are sealed by the waterproof film.

In one or more embodiments, a second surface of the first film is configured to be attached to a fabric wear, wherein the fabric wear includes at least one of underpants, an underwear, a knee pad, a wrist guard, an elbow pad, sport pants and a pain patch, and the second surface of the first film is an opposite surface of the first surface of the first film.

In one or more embodiments, the processing module is configured to be fixed to a fabric wear or separated from the fabric wear.

In one or more embodiments, the sensing module includes a first conductive fiber sensing portion arranged in a first sensing region of the textile and configured to generate a first sensing signal of the physiological sensing signal. The processing module is further configured to determine whether the physiological event is an erection event according to the change in the first sensing signal detected in the first sensing region by the first conductive fiber sensing portion.

In one or more embodiments, the sensing module further includes a second conductive fiber sensing portion arranged in a second sensing region of the textile, coupled to the processing module, and configured to generate a second sensing signal of the physiological sensing signal. The processing module is further configured to determine whether the physiological event is a sleep event according to the change in the second sensing signal detected in the second sensing region by the second conductive fiber sensing portion.

In one or more embodiments, the physiological sensing apparatus further includes a gravity sensor (G sensor) coupled to the processing module and configured to generate a three-axis signal. When determining that the physiological event is a sleep event, the processing module is further configured to determine whether the three-axis signal is received and determine a sleep posture state of the sleep event according to the three-axis signal, wherein the sleep posture state includes a back lying state, a left lying state, a right lying state, a front lying state, a bed leaving state and a walking state.

In one or more embodiments, the sensing module further includes a third conductive fiber sensing portion arranged in a third sensing region of the textile and coupled to the processing module. The third conductive fiber sensing portion is configured to generate a third sensing signal of the physiological sensing signal for the processing module to determine at least one of a heart rhythm state, a breathing state and a body temperature state of the physiological event.

In one or more embodiments, the processing module is further configured to determine the heart rhythm state of the physiological event according to the change in the third sensing signal; and when determining that the change in the third sensing signal is abnormal, the processing module records the heart rhythm state as an abnormal state.

In one or more embodiments, the processing module is further configured to determine the breathing state of the physiological event according to the change in the third sensing signal; and when determining that the change of the third sensing signal is abnormal, the processing module records the breathing state as an abnormal state.

In one or more embodiments, the processing module is further configured to determine the body temperature state of the physiological event according to the change in the third sensing signal; and when determining that the change of the third sensing signal is abnormal, the processing module records the body temperature state as an abnormal state.

In one or more embodiments, the physiological sensing apparatus further includes a storage medium coupled to the processing module. The storage medium is configured to store the physiological event associated with the physiological signal for the processing module to determine the current physiological event according to the change in the physiological signal.

In one or more embodiments, the physiological sensing apparatus further includes a wireless transmission module coupled to the processing module. The wireless transmission module is configured to transmit the physiological event to an electronic device, such that the electronic device displays a message on the physiological event.

In one or more embodiments, the physiological sensing apparatus further includes a battery module and a charging module that is coupled to the processing module and the charging module. The charging module is configured to charge the battery module, such that the battery module provides power to the processing module.

According to another embodiment, a physiological sensing method is disclosed and applicable to a physiological sensing apparatus, wherein the physiological sensing apparatus includes a sensing module and a processing module. The sensing module includes a sensor element and a first film. A first surface of the first film includes a conductive ink pattern, and a first surface of the sensor element is superimposed on the first surface of the first film, such that the conductive ink pattern is in contact with the sensor element. The physiological sensing method includes the following steps: generating, by the sensing module arranged on a textile, a physiological sensing signal; transmitting the physiological sensing signal to the processing module; analyzing, by the processing module, a change in the physiological sensing signal; and determining, according to the change, a physiological event corresponding to the physiological sensing signal.

In one or more embodiments, a first conductive fiber portion of the sensing module is arranged in a first sensing region of the textile. The physiological sensing method further includes: generating, by the first conductive fiber portion, a first sensing signal of the physiological sensing signal; and determining, according to the change in the first sensing signal, whether the physiological event is an erection event.

In one or more embodiments, a second conductive fiber portion of the sensing module is arranged in a second sensing region of the textile. The physiological sensing method further includes: generating, by the second conductive fiber portion, a second sensing signal of the physiological sensing signal; and determining, according to the change in the second sensing signal, whether the physiological event is a sleep event.

In one or more embodiments, the physiological sensing method further includes receiving a three-axis signal by the processing module, and when determining that the physiological event is the sleep event, determining a sleep posture state of the sleep event according to the three-axis signal, wherein the sleep posture state includes a back lying state, a left lying state, a right lying state, a front lying state, a bed leaving state and a walking state.

In one or more embodiments, a third conductive fiber portion of the sensing module is arranged in a third sensing region of the textile. The physiological sensing method further includes generating a third sensing signal of the physiological sensing signal by the third conductive fiber portion for the processing module to determine at least one of a heart rhythm state, a breathing state and a body temperature state of the physiological event.

In one or more embodiments, the step of determining the physiological event by the processing module according to the third sensing signal further includes determining the heart rhythm state of the physiological event according to the change in the third sensing signal; and when determining that the change in the third sensing signal is abnormal, recording the heart rhythm state as an abnormal state.

In one or more embodiments, the step of determining the physiological event by the processing module according to the third sensing signal further includes determining the breathing state of the physiological event according to the change in the third sensing signal; and when determining that the change in the third sensing signal is abnormal, recording the breathing state as an abnormal state.

In one or more embodiments, the step of determining the physiological event by the processing module according to the third sensing signal further includes determining the body temperature state of the physiological event according to the change in the third sensing signal; and when determining that the change in the third sensing signal is abnormal, recording the body temperature state as an abnormal state.

In one or more embodiments, the physiological sensing method further includes transmitting the physiological event to an electronic device, such that the electronic device displays a message on the physiological event.

According to another embodiment, a physiological information service system is disclosed. The physiological information service system includes a physiological sensing apparatus and a first electronic device. The physiological sensing apparatus is detachably arranged on a textile. The physiological sensing apparatus includes a sensing module and a processing module. The sensing module is configured to generate a physiological sensing signal, and the sensing module includes a sensor element and a first film. The sensor element is configured to detect the physiological sensing signal. A first surface of the first film includes a conductive ink pattern, and a first surface of the sensor element is superimposed on the first surface of the first film such that the conductive ink pattern is in contact with the sensor element. The processing module is coupled to the sensing module and configured to receive the physiological sensing signal to determine a physiological event corresponding to a change in the physiological sensing signal. The first electronic device is configured to establish a network connection with the physiological sensing apparatus through a gateway. The electronic device receives the physiological event through the network connection to prompt a detection warning message.

In one or more embodiments, the gateway is in communication connection with a server. The electronic device is configured to receive the physiological event through the server to prompt the detection warning message according to the physiological event.

### BRIEF DESCRIPTION OF DRAWINGS

FIG. 1A illustrates a schematic diagram of a physiological sensing apparatus arranged on a textile according to one or more embodiments of the present disclosure;

FIG. 1B illustrates a schematic structural diagram of a sensing module according to some embodiments of the present disclosure;

FIG. 1C illustrates a schematic structural diagram of a plurality of sensor elements of FIG. 1B;

FIG. 1D illustrates a schematic structural diagram of a sensing module according to some other embodiments of the present disclosure;

FIG. 1E illustrates a schematic structural diagram of a sensing module according to some other embodiments of the present disclosure;

FIG. 2 illustrates a functional block diagram of a physiological sensing apparatus according to some embodiments of the present disclosure;

FIG. 3 illustrates a schematic diagram of application of the physiological sensing apparatus of FIG. 2 to a textile according to another embodiment of the present disclosure;

FIG. 4A illustrates a flowchart of steps of a physiological sensing method according to some embodiments of the present disclosure;

FIG. 4B illustrates a flowchart of steps of a physiological sensing method according to some other embodiments of the present disclosure;

FIG. 5 is a schematic diagram of an environment of a physiological information service system according to some embodiments of the present disclosure;

FIG. 6 is a schematic diagram of an environment of a physiological information service system according to some embodiments of the present disclosure.

### DETAILED DESCRIPTION

The following disclosure provides many different embodiments or examples to implement different features of the present invention. The specific examples of components and arrangements are described below to simplify the present invention. Of course, these examples are only exemplary and are not restrictive. For example, in the following description, a first feature being formed above or on a second feature may include one or more embodiments in which the first feature and the second feature are formed in direct contact, and may also include one or more embodiments in which an additional feature is formed between the first feature and the second feature such that the first feature and the second feature are not in direct contact. In addition, symbols and/or letters of components may be repeated in various examples of the present invention. This repetition is for the purpose of conciseness and clearness, and it does not indicate the relationship between the various embodiments and/or configurations discussed.

Further, in order to facilitate the description, terms of spatial relation (such as “beneath”, “below”, “lower”, “above”, “higher” and the like) may be used herein to describe the relationship between one component or feature and the other component(s) or feature(s) as shown in the drawings. In addition to the orientations depicted by the drawings, the terms of spatial relation are intended to include different orientations of a device in use or operation. The device may be oriented in other ways (rotated 90 degrees or in other orientations) and the terms of spatial relation used herein can also be such interpreted.

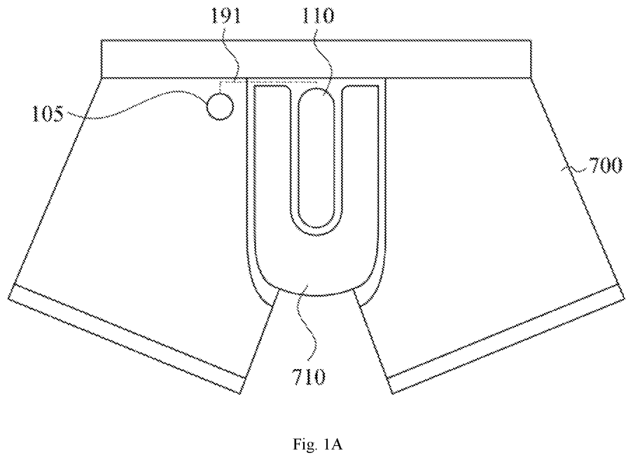

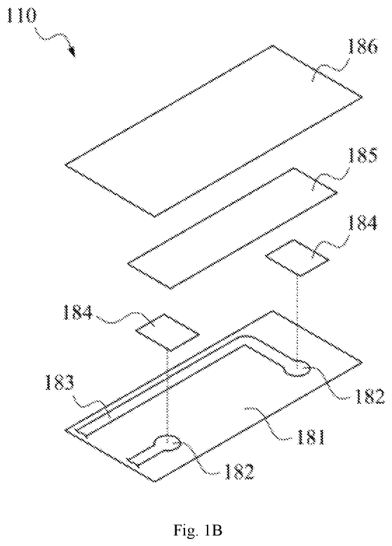

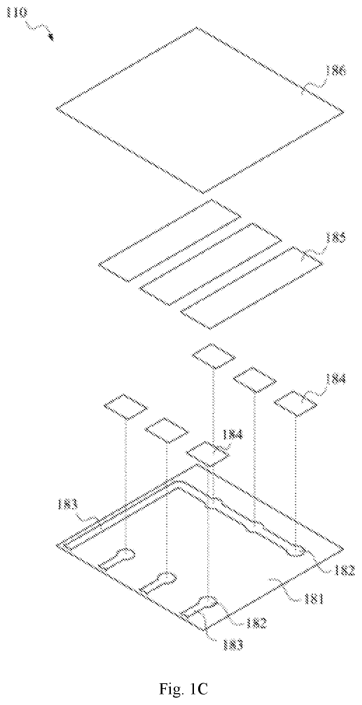

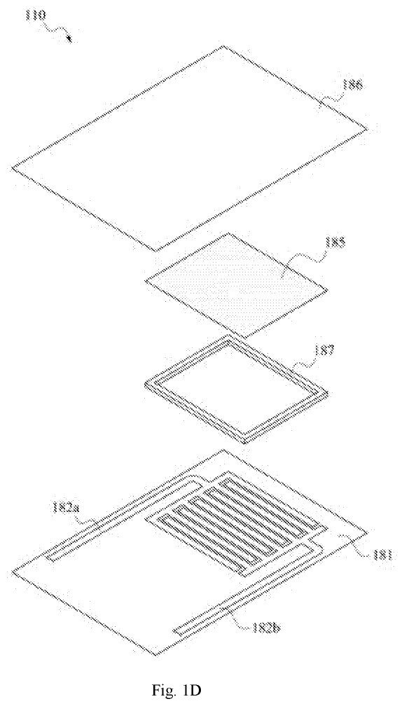

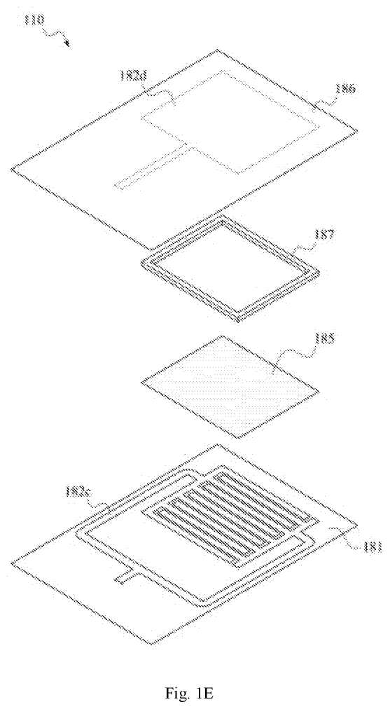

Referring to FIG. 1A, a schematic diagram of a physiological sensing apparatus arranged on a textile 700 according to one or more embodiments of the present disclosure is illustrated. The physiological sensing apparatus has a housing 105 and a sensing module 110. The housing 105 includes a plurality of electronic components therein, and details of the electronic components will be described later. The electronic components inside the housing 105 are connected to the sensing module 110 on the textile 700 via a wire 191. In one or more embodiments, the housing 105 may be similar to a macaron in appearance and has an accommodating space such that the electronic components of the physiological sensing apparatus may be arranged in the accommodating space. It is worth mentioning that the housing 105 may be, but is not limited to, plastic, acrylic, textile, etc. Any one capable of accommodating the electronic components and connected to the sensing module 110 falls into the scope of the present disclosure.

In some embodiments, the textile 700 may be, but is not limited to, underwear worn on the body, a mattress, a seat cushion, a back cushion, fabric wear, etc. The fabric wear may be, but are not limited to underpants, an underwear, a knee pad, a wrist guard, an elbow pad, sport pants, a pain patch, etc. Underpants are taken as an example in FIG. 1A, and the present disclosure is not limited thereto.

The housing 105 is detachably arranged on the textile 700 such that the electronic components except the sensing module 110 in the physiological sensing apparatus may be removed along with the housing 105. In some cases, the user can remove the physiological sensing apparatus 100 from the textile 700 such that the textile 700 has only the sensing module 110 and is worn by the user as common clothing.

The sensing module 110 may be attached (e.g., hot-pressed, pasted, etc.), sewed or interleaved to the textile 700 such that the skin may directly contact the sensing module 110 when the user is wearing the textile 700. In one or more embodiments, the sensing module 110 is a fabric sensor or sensor array of conductive fibers made from a conductive material with low resistance. When the user wears the textile 700, the sensing module 110 acquires a physiological sensing signal due to the contact with human skin. In some embodiments, the sensing module 110 includes a conductive film printed with an elastic conductive ink.

The physiological sensing signal detected by the sensing module 110 may be transmitted to the physiological sensing apparatus through the wire 191. The physiological sensing apparatus resolves the physiological sensing signal and analyzes a change pattern of the physiological sensing signal. After resolving the change pattern, the physiological sensing apparatus determines which state (e.g., erection, sitting posture, urine leakage, heart rhythm value, breathing frequency, body temperature, pregnant women's fetal movement, etc.) the body of the user wearing the textile 700 is currently in.

In one or more embodiments, only the sensor or sensing array capable of sensing male genital erection is arranged on the textile 700, but the present disclosure is not limited thereto. In another embodiment, the textile 700 may also be provided with more than one conductive fiber sensor or sensing array to increase the type or pattern of the physiological signal that can be captured, thereby improving convenience for the user to monitor physiological information.

Hereinafter, FIGS. 1B-1E will illustrate the internal structure of the sensing module 110. In order to facilitate description, FIGS. 1B-1E show structural relationships between the internal components of the sensing module 110. In fact, the sensing module 110 is a flat shape formed by laminating layers together.

Referring to FIG. 1B, a schematic structural diagram of a sensing module 110 according to some embodiments of the present disclosure is illustrated. As shown in FIG. 1B, the sensing module 110 includes a first film 181, a conductive film 184, a sensor element 185, and a second film 186.

A surface (e.g., an upward surface shown in FIG. 1B) of the first film 181 is printed with a conductive ink pattern. The conductive ink pattern includes an electrode pattern 182 and a circuit pattern 183. The conductive film 184 covers the electrode pattern 182 such that the conductive film 184 is in contact with the electrode pattern 182. The conductive film 184 may also be configured to fix the electrode pattern 182 to avoid the movement of the electrode pattern 182 on the first film 181. The conductive ink pattern may be, but is not limited to, conductive silver paste, metal powder or other conductive filler with high electric conductivity. The first film 181 may be, but is not limited to, an elastic waterproof film.

In some embodiments, the sensing module 110 includes one or more sensor elements 185.

A surface (e.g., a downward surface shown in FIG. 1B) of the sensor element 185 is superimposed on the surface (e.g., the upward surface shown in FIG. 1B) of the first film 181, such that the electrode pattern 182 is in contact with the sensor element 185. In one or more embodiments, the electrode pattern 182 and the circuit pattern 183 form a circuit that can transmit a sensing signal. When the sensor element 185 receives a physiological sensing signal, the physiological sensing signal is transmitted to the physiological sensing apparatus via the electrode pattern 182 and the circuit pattern 183.

The sensor element 185 may be, but is not limited to, a conductive fabric. The sensor element 185 is, for example, a semiconductor sensor (a temperature sensor, a pressure sensor, etc.), a semiconductor sensing module (a Bluetooth module, a wireless transmission module), etc.

The second film 186 is arranged on the other surface (e.g., an upward surface shown in FIG. 1B) of the sensor element 185 such that the second film 186 covers the sensor element 185 and the circuit pattern 183. In one or more embodiments, the second film 186 is configured to fix or protect the components in the sensing module 110 to prevent the components from being exposed or subjected to direct external force damage. The second film 186 may be, but is not limited to, a plastic film, a fabric, a waterproof material, thermoplastic polyurethane (TPU), and the like. In some other embodiments, the sensing module 110 may be arranged in a closed object (e.g., in a cushion), without including the second film 186.

In some embodiments, a waterproof film is arranged above the first film 181 and the sensor element 185 such that the first film 181 and the sensor element 185 are sealed by the waterproof film.

Referring to FIG. 1C, a schematic structural diagram of using a plurality of sensor elements 185 of FIG. 1B is illustrated. Hereinafter, only the differences from FIG. 1B are described, the remaining same numerals are referred to the foregoing description, and details are not described herein again. The conductive ink pattern printed on the first film 181 of the sensing module 110 includes a plurality of electrode patterns 182 and a plurality of circuit patterns 183, wherein the electrode patterns 182 are connected by the circuit patterns 183 to form a conductive circuit. In FIG. 1C, the sensing module 110 includes three sensor elements 185, and each sensor element 185 is in contact with two electrode patterns 182, such that the physiological sensing signals generated by the sensor elements 185 can be transmitted to the physiological sensing apparatus by the circuit patterns 183.

Referring to FIG. 1D, a schematic structural diagram of a sensing module 110 according to some other embodiments of the present disclosure is illustrated. In FIG. 1D, the components in the sensing module 110 include, from bottom to top, a first film 181, a first electrode pattern 182a, a second electrode pattern 182b, a spacer 187, a sensor element 185, and a second film 186. The components of the same numerals in FIG. 1D as in FIG. 1B are described as above.

In one or more embodiments, the first electrode pattern 182a and the second electrode pattern 182b are of comb structures. For example, the comb structure of the first electrode pattern 182a includes a plurality of strip-shaped resistors arranged parallel to each other, and ends of the strip-shaped resistors in the same direction are connected to each other, such that the strip-shaped resistors form a parallel relationship. The ends of the strip-shaped resistors in the other direction are not connected to each other such that the entire first electrode pattern 182a exhibits a pattern similar to a comb (or a rake). The second electrode pattern 182b is also the same.

In one or more embodiments, the strip-shaped resistors in the comb structures of the first electrode pattern 182a and the second electrode pattern 182b are arranged alternately to each other such that the strip-shaped resistors of the first electrode pattern 182a are arranged in gaps between the strip-shaped resistors of the second electrode pattern 182b. Similarly, the strip-shaped resistors of the second electrode pattern 182b are arranged in gaps between the strip-shaped resistors of the first electrode pattern 182a.

In this way, each of the strip-shaped resistors can be considered as a small resistor. After these small resistors are connected in parallel, the resistance value obtained by the first electrode pattern 182a can be more linear. The second electrode pattern 182b is also the same. On the other hand, each small resistor is more easily contacted when on the same plane, and since the contacted plane portion can be distinguished more delicately, the small resistors at different positions may generate sensing signals more accurately to present force more completely. Further, in the comb structures, the coating material may be changed on the small resistors according to actual requirements, to correspondingly change the electric conductivity or the impedance in different application occasions (such as occasions of different intensity or sensitivity of sensing force).

In one or more embodiments, the first film 181 is printed with the first electrode pattern 182a and the second electrode pattern 182b. One end of the first electrode pattern 182a is superimposed with the sensor element 185. One end of the second electrode pattern 182b is superimposed with the sensor element 185. After the sensor element 185 generates a physiological sensing signal, the physiological sensing signal is transmitted to the physiological sensing apparatus via the first electrode pattern 182a and the second electrode pattern 182b.

A surface (e.g., an upward surface shown in FIG. 1D) of the sensor element 185 may be or may not be provided with a spacer 187. The spacer 187 is an approximately rectangular hollow column, and the peripheral profile of the spacer 187 is superimposed on the peripheral profile of the sensor element 185. The spacer 187 may be, but is not limited to, a film, foam, a cloth having a thickness, a thermal melt adhesive, or the like of a non-conductive material. In one or more embodiments, the sensing module 110 may be selectively provided with the spacer 187. When the sensing module 110 is provided with the spacer 187, the sensor element 185 is respectively spaced from the first electrode pattern 182a and the second electrode pattern 182b by a small distance, to adjust the sensitivity of the physiological sensing signal detected by the sensor element 185. When the sensor element 185 is a fabric sensor, a protruding fabric portion (not shown) may be designed through the fabric structure (e.g., yarn packs, towel fibers, etc.) to produce the isolation effect without the configuration of the spacer 187, and the sensitivity of the physiological sensing signal is adjusted by this structure design. In another embodiment, according to actual implementation requirements, when the sensor element 185 is a fabric sensor, the sensing module 110 may also be provided with the spacer 187 to adjust the sensitivity of the physiological sensing signal to different degrees.

Referring to FIG. 1E, a schematic structural diagram of a sensing module 110 according to some other embodiments of the present disclosure is illustrated. In FIG. 1E, the components in the sensing module 110 include, from bottom to top, a first film 186, a first electrode pattern 182c, a sensor element 185, a spacer 187, a second electrode pattern 182d, and a second film 186. In FIG. 1E, the same numerals as in FIG. 1D are referred to the foregoing description, and details are not described herein again.

It is worth mentioning that in some embodiments, the spacer 187 in FIG. 1E may be arranged above the sensor element 185 such that the spacer 187 is between the sensor element 185 and the second electrode pattern 182d, and the sensor element 185 is spaced from the second electrode pattern 182d by a small distance, to adjust the sensitivity of the physiological sensing signal detected by the sensor element 185. Alternatively, the spacer 187 may be arranged below the sensor element 185 such that the spacer 187 is between the sensor element 185 and the first electrode pattern 182c, and the sensor element 185 is spaced from the first electrode pattern 182a by a small distance to adjust the sensitivity of the physiological sensing signal detected by the sensor element 185. In one or more embodiments, the sensing module 110 may be selectively provided with the spacer 187. When the sensor element 185 is a fabric sensor, the fabric structure of the sensor element 185 may be designed with a protruding fabric portion (not shown) to produce the isolation effect between the sensor element 185 and the first electrode pattern 182c without the configuration of the spacer 187, and the sensitivity of the physiological sensing signal can be adjusted by this structure design. In another embodiment, when the sensor element 185 is a fabric sensor with a protruding fabric portion (not shown), the sensing module 110 may also be provided with the spacer 187, and details are described as above.

It is worth mentioning that the protruding fabric portion structure may be, but is not limited to, an upward protruding structure, a downward protruding structure, a regular protruding structure, an irregular protruding structure, or the like. The present disclosure is not intended to limit the actual structure and/or shape of the protruding fabric portion structure. Any structure that can partially/fully increase the distance between the sensor element 185 and the first electrode pattern 182a and the second electrode pattern 182b, or partially/fully increase the distance between the sensor element 185 and the first electrode pattern 182c and/or the second electrode pattern 182d, falls into the scope of the present disclosure.

In one or more embodiments, the first electrode pattern 182c may be of a paired comb structure. As shown in FIG. 1E, the first electrode pattern 182c includes a comb structure that opens toward the left (the first direction) and a comb structure that opens toward the right (the second direction), wherein the two comb structures are arranged alternately with each other, and transmit the sensing signal via the circuit that connects the resistors in parallel. The description of the comb structure is referred to the foregoing content, and details are not described herein again.

A surface (e.g., an upward surface shown in FIG. 1E) of the first film 181 is printed with a first electrode pattern 182c. The first electrode pattern 182c is partially in contact with a surface (e.g., a downward surface shown in FIG. 1E) of the sensor element 185. The other surface (e.g., an upward surface shown in FIG. 1E) of the sensor element 185 is provided with the spacer 187.

A surface (e.g., a downward surface shown in FIG. 1E) of the second film 186 is printed with a second electrode pattern 182d. When the downward surface of the second film 186 is superimposed with the upward surface of the first film 181, the second electrode pattern 182d is in contact with the first electrode pattern 182c, such that the physiological sensing signal generated by the sensor element 185 may be transmitted to the physiological sensing apparatus via the circuit formed by the first electrode pattern 182c and the second electrode pattern 182d. In one or more embodiments, at least a part of the first electrode pattern 182c is in contact with at least a part of the second electrode pattern 182d to form an electrical circuit between the sensor element 185 and the physiological sensing apparatus.

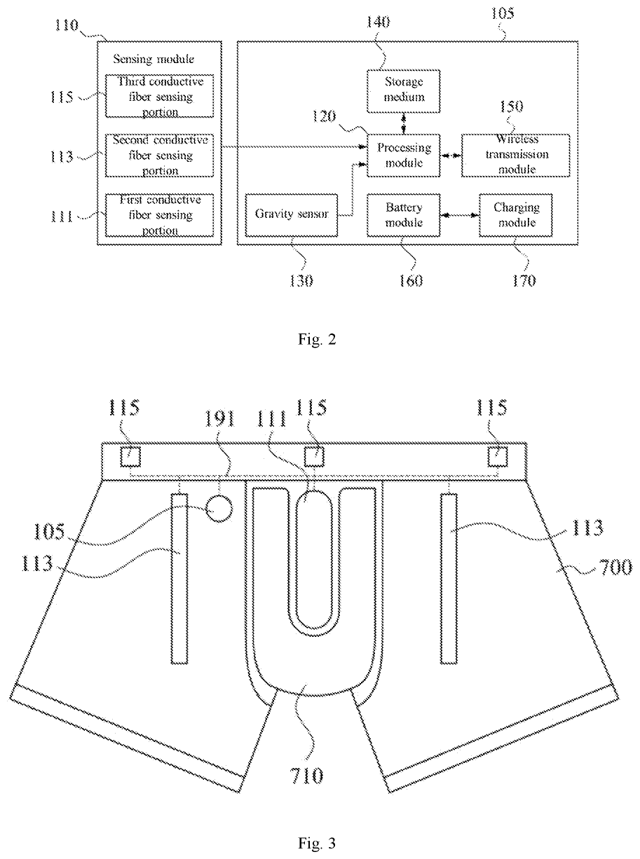

Referring to FIG. 2, a functional block diagram of a physiological sensing apparatus 100 according to some embodiments of the present disclosure is illustrated. As shown in FIG. 2, the physiological sensing apparatus 100 includes a sensing module 110, a processing module 120, a gravity sensor (G sensor) 130, a storage medium 140, a wireless transmission module 150, a battery module 160, and a charging module 170.

In one or more embodiments, the physiological sensing apparatus 100 has a housing 105, wherein the processing module 120, the G sensor 130, the storage medium 140, the wireless transmission module 150, the battery module 160, and the charging module 170 are accommodated in the housing 105. The sensing module 110 is connected to a circuit contact (not shown) on the housing 105 of the physiological sensing apparatus 100 by a wire 191 to be connected to the processing module 120. The processing module 120 may be a central processor unit (CPU), a system on chip (SoC), an application processor, an audio processor, a digital signal processor, or a specific functional processing chip or controller.

The sensing module 110 is coupled to the processing module 120. In one or more embodiments, the sensing module 110 is configured to detect a mechanical force and convert it into an electrical signal. Referring to FIG. 1A again, when the user wears the textile 700 (i.e., underpants), the region of a liner 710 is the position of a male genital organ. When the male genital organ erects, the male genital organ is in direct contact with the region of the liner 710 to generate a physiological sensing signal. Accordingly, the sensing module 110 determines the change in a resistance value to obtain the physiological sensing signal, and determines that the male genital organ currently wearing the underpants is in an erection state. It is worth mentioning that the liner 710 may be a comfortable fibrous material having a thickness. When the sensing module 110 detects the erection of the male genital organ, the signal may be automatically introduced into the comfortable region of the liner 710.

In one or more embodiments, the sensing module 110 may be, but is not limited to a conductive fiber sensor or sensor array of a thermistor, a force sensing resistor or a varistor. Therefore, the sensing module 110 may capture different types of sensing signals. As shown in FIG. 2, the sensing module 110 includes a first conductive fiber sensing portion 111, a second conductive fiber sensing portion 113, and a third conductive fiber sensing portion 115. Each conductive fiber sensing portion may detect different types of physiological sensing signals respectively for the processing module 120 to determine which physiological state the user is currently in.

Referring to FIG. 2 again, the storage medium 140 of the physiological signal sensing device 100 is coupled to the processing module 120. The storage medium 140 is configured to store physiological events associated with multiple or different physiological signals. The processing module 120 may determine the current physiological event later according to the change or pattern of the physiological signal. The storage medium 140 may be a Random Access Memory (RAM) or a non-volatile memory (such as a Flash memory), a Read Only Memory (ROM), a Hard Disk Drive (HDD), a Solid State Drive (SSD) or an optical memory, etc.

The wireless transmission module 150 of the physiological signal sensing device 100 is coupled to the processing module 120. The wireless transmission module 150 is configured to transmit the physiological event determined by the processing module 120 to an electronic device (not shown) via a wireless communication protocol. Thus, the electronic device only needs to be provided with an application or a transmission protocol corresponding to the physiological signal sensing device 100, to display relevant information about the physiological event. The wireless transmission module 150 may be a communication chip supporting Global System for Mobile communication (GSM), Long Term Evolution (LTE), Worldwide interoperability for Microwave Access (WiMAX), Wireless Fidelity (Wi-Fi), Bluetooth technology or wired networks.

The battery module 160 of the physiological signal sensing device 100 is coupled to the processing module 120 and the charging module 170. The charging module 170 is configured to charge the battery module 160 such that the battery module 160 can provide power to all electronic components of the physiological signal sensing device 100. In one or more embodiments, the physiological signal sensing device 100 may be removed and placed on a charging base connected to the mains to receive the power of the mains through the charging module 170 such that the battery module 160 is charged.

Referring to FIG. 3, a schematic diagram of application of the physiological sensing apparatus 100 of FIG. 2 to a textile 700 according to another embodiment of the present disclosure is illustrated. As shown in FIG. 3, the textile 700 is provided with the first conductive fiber sensing portion 111 in a first sensing region (e.g., the male genital part). The first conductive fiber sensing portion 111 is connected to the housing 105 of the physiological sensing apparatus by the wire 191. As described above, the first conductive fiber sensing portion 111 is configured to contact the skin to generate a sensing signal. The sensing signal is transmitted to the processing module 120 by the wire 191. The processing module 120 determines whether or not an erection is present according to the change in the sensing signal.

As shown in FIG. 3, the textile 700 is provided with the second conductive fiber sensing portion 113 in a second sensing region (e.g., a hip part). The second conductive fiber sensing portion 113 is connected to the physiological sensing apparatus 100 by the wire 191. In one or more embodiments, the second conductive fiber sensing portion 113 is configured to detect a mechanical force and convert it into an electrical signal. For example, when the user is lying on the bed, the second conductive fiber sensing portion 113 contacts the skin of the hip and correspondingly generates a sensing signal. The sensing signal is transmitted to the processing module 120 by the wire 191. The processing module 120 detects the activity of lying in bed according to the change in the sensing signal to generate a corresponding sleep indicator or a lying posture signal, and determines whether it is a sleep event or a lying event. In one or more embodiments, the number of user actions detected is designed as a sleep indicator. If the detected number of user's actions for a long time is low (for example, ten or less actions are detected within one hour), it represents a high sleep indicator, and a sleep state is determined.

Referring to FIG. 2 again, the G sensor 130 of the physiological sensing apparatus 100 is coupled to the processing module 120. The G sensor 130 is configured to generate a three-axis signal. When the processing module 120 determines that the user is currently in a sleep state, it further determines whether the three-axis signal is received. The three-axis signal is used to determine a sleep posture state of the user by the processing module 120. For example, the processing module 120 determines, according to the three-axis signal, that the sleep posture of the user is back lying, left lying, right lying, front lying, bed leaving, walking, or the like.

As shown in FIG. 3, the textile 700 is provided with the third conductive fiber sensing portion 115 in a third sensing region (e.g., a waist part). The third conductive fiber sensing portion 115 is connected to the physiological sensing apparatus 100 by the wire 191. In one or more embodiments, the third conductive fiber sensing portion 115 can detect a plurality of different types of physiological signals. For example, the third conductive fiber sensing portion 115 may be a conductive fiber sensor or sensor array of a varistor. At this time, the third conductive fiber sensing portion 115 may be used as a heartbeat detection electrode, which is in contact with the skin and correspondingly generates a sensing signal. The sensing signal is transmitted to the processing module 120 by the wire 191. The processing module 120 determines a heart rhythm state of the user according to the change in the sensing signal (e.g., the change in the voltage or potential) to obtain a heart rhythm or a pulse rate of the user.

The third conductive fiber sensing portion 115 may also be a conductive fiber sensor or sensor array of a thermistor. At this time, the third conductive fiber sensing portion 115 is in contact with the skin and correspondingly generates a sensing signal. The sensing signal is transmitted to the processing module 120 by the wire 191. The processing module 120 measures a body temperature of the user according to the change in the sensing signal (e.g., the change in the resistance value).

The third conductive fiber sensing portion 115 may also be a conductive fiber sensor or sensor array of a force sensing resistor. The third conductive fiber sensing portion 115 is in contact with the skin and correspondingly generates a sensing signal. The sensing signal is transmitted to the processing module 120 by the wire 191. The processing module 120 measures the number or frequency of the user's breathing according to the change in the sensing signal (e.g., a mechanical stress change caused by the rise and fall of the waist).

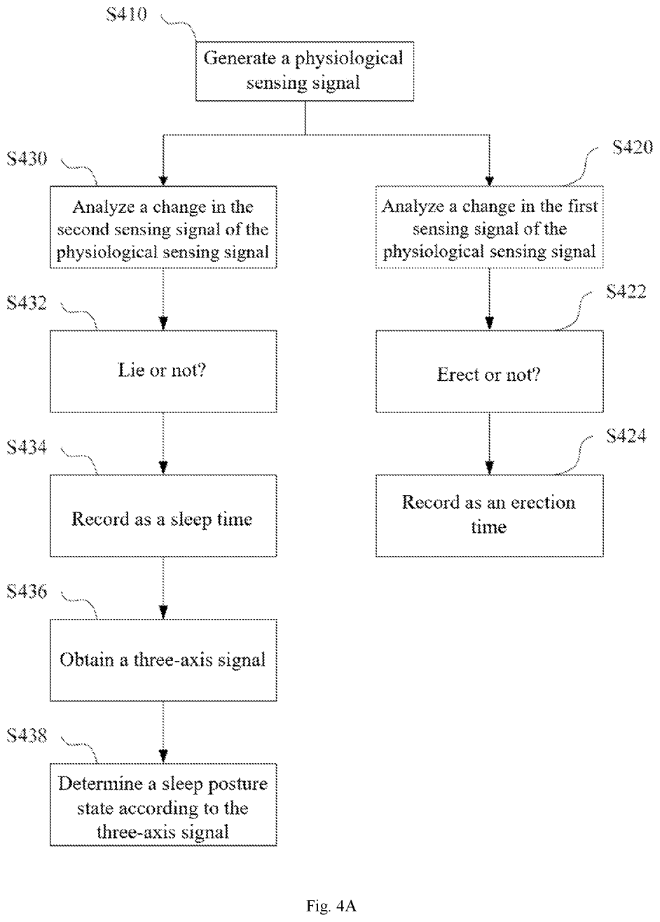

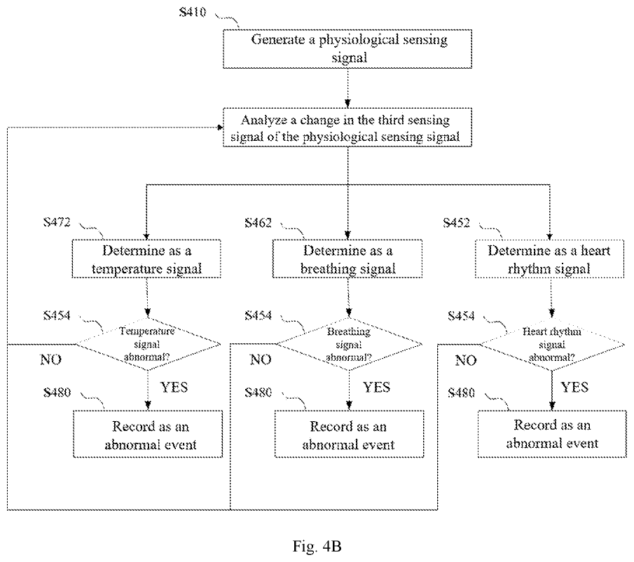

Referring to FIG. 4A, a flowchart of steps of a physiological sensing method according to some embodiments of the present disclosure is illustrated. The physiological sensing method is suitable for the physiological sensing apparatus 100 shown in FIG. 2. In step S410, after the sensing module 110 contacts the user's skin, a physiological sensing signal is generated. The physiological sensing signal may be, but is not limited to, an erection signal, a sleep lying signal, a vital sign signal, etc. In step S420, the processing module 120 receives and analyzes the change in the sensing signal about erection. For example, when the male genital organ erects and contacts the sensing module 110, the activity signal change in the male genital organ is analyzed according to the sensing signal. In step S422, whether or not an erection state is present is determined. If erection is determined according to the activity signal change, then in step S424, the time of occurrence of the sensing signal and the erection event are recorded in the storage medium 140.

In step S430, the processing module 120 receives and analyzes the change in the sensing signal about lying in bed. For example, when the user's hip is in contact with the sensing module 110, the sensing signal about a lying posture is received. Next, in step S432, whether or not the lying posture is present is determined according to the sensing signal. If the lying in bed is determined, then in step S434, the time of occurrence of the sensing signal and the sleep event are recorded in the storage medium 140. Next, the processing module 120 can further determine details of the lying posture, for example, in step S436, the G sensor 130 generates a three-axis signal by which the processing module 120 can determine the user's body moving state, lying turnover action, or the like. In step S438, the processing module 120 determines the sleep posture state of the user according to the three-axis signal.

Referring to FIG. 4B, a flowchart of steps of a physiological sensing method according to some other embodiments of the present disclosure is illustrated. The physiological sensing method is suitable for the physiological sensing apparatus 100 shown in FIG. 2. In step S440, the processing module 120 receives and analyzes the change in the sensing signal about a vital sign. The vital sign signal may be, but is not limited to, a heart rhythm signal, a breathing signal, a body temperature signal, and the like.

In step S452, a heart rhythm signal is determined from the resistance value or change mode of the sensing signal. Next, in step S454, the processing module 120 determines whether the change in the heart rhythm signal is abnormal. If the heart rhythm signal is determined to be abnormal, step S480 is performed to record a heart rhythm abnormality event in the storage medium 140.

In step S462, a breathing signal is determined from the resistance value or change mode of the sensing signal. Next, in step S464, the processing module 120 determines whether the change in the breathing signal is abnormal. If the breathing signal is determined to be abnormal, step S480 is performed to record a breathing abnormality event in the storage medium 140.

In step S472, a temperature signal is determined from the resistance value or change mode of the sensing signal. Next, in step S474, the processing module 120 determines whether the change in the temperature signal is abnormal. If the temperature signal is determined to be abnormal, step S480 is performed to record a body temperature abnormality event in the storage medium 140.

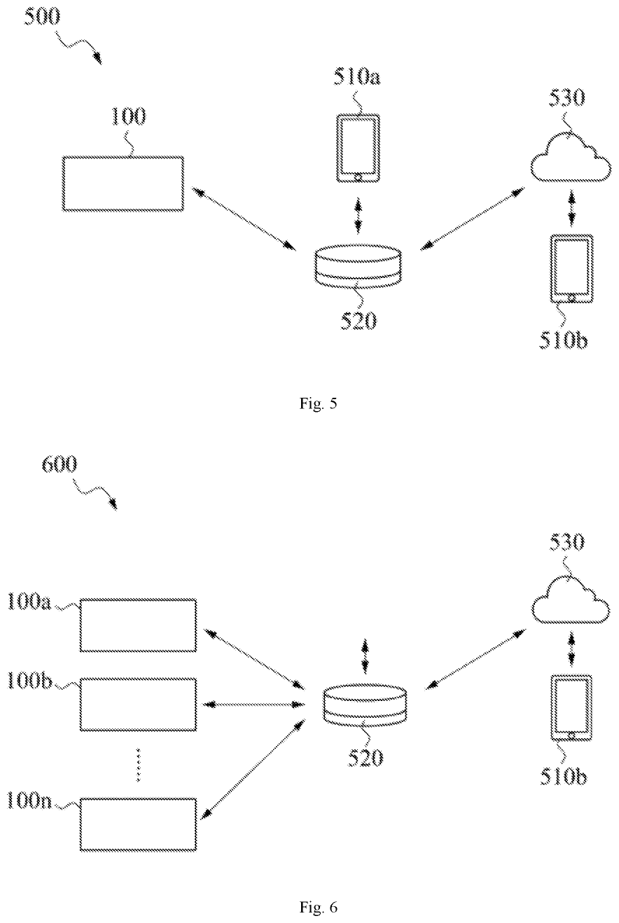

Referring to FIG. 5, a schematic diagram of an environment of a physiological information service system 500 according to some embodiments of the present disclosure is illustrated. As shown in FIG. 5, the physiological information service system 500 includes a physiological sensing apparatus 100 and an electronic device 510a. The physiological sensing apparatus 100 is configured to generate a relevant physiological event, and details are described above. Wireless communication is established between the physiological sensing apparatus 100 and a gateway 520.

In one or more embodiments, the electronic device 510a is connected with the gateway 520 by a wireless network, for example, the both are in the same wireless network environment. The electronic device 510a can be wirelessly connected to obtain the physiological event of the physiological sensing apparatus 100 through the gateway 520, thereby prompting a detection warning message.

In another embodiment, the electronic device 510b can access the information on a server 530 via a wired network or a wireless network. For example, the physiological sensing apparatus 100 can upload the physiological event and/or a relevant physiological sensing signal to the server 530. The user can operate the electronic device 510b to access the information on the server 530 to understand the physiological state at each time point.

Referring to FIG. 6, a schematic diagram of an environment of a physiological information service system 600 according to some other embodiments of the present disclosure is illustrated. As shown in FIG. 6, the physiological information service system 600 includes a plurality of physiological sensing apparatus 100a to 100n and an electronic device 510c. The physiological sensing apparatus 100a to 100n may be arranged on different textiles and worn by different users. A wireless communication connection is respectively established between the physiological sensing apparatus 100a to 100n and the gateway 520. As described above, the physiological sensing apparatus 100a to 100n respectively upload physiological events or relevant physiological sensing signals to the server 530. A relevant user can operate the electronic device 510c to access the information on the server 530 via a wired network or a wireless network to understand physiological states of respective users wearing the physiological sensing apparatus 100a to 100n at different time points.

As shown in FIGS. 5 and 6, the electronic devices 510a to 510c can obtain more applications and services, such as erection detection and warning, lying posture detection and warning, sleep quality detection and warning, bed leaving warning and detection, heartbeat detection and warning, breathing detection and warning, body temperature detection and warning, fall detection and warning, etc., through the physiological information service systems 500 and 600, to provide more diverse services for human life.

The electronic devices 510a to 510c may be, but are not limited to, portable electronic devices, mobile phones, tablet computers, personal digital assistants (PDA), wearable devices or notebooks.

In summary, an administrator or a healer who is going to monitor one or more users may use the physiological sensing apparatus 100, the physiological sensing method, and the physiological information service system 500, 600 of the present disclosure to master the physiological state of each user, so as to provide a better life quality. In addition, because the physiological sensing apparatus 100 is small and portable, the textile 700 is more convenient to wear, the physiological sensing signal can be detected from time to time, and the user's discomfort can be reduced.

The features of several embodiments are summarized above, so that a person skilled in the art can better understand the present invention. A person skilled in the art can easily use the present invention as a basis for designing or modifying other processes and structures to achieve the same purposes and/or the same advantages of the embodiments described herein. A person skilled in the art could also recognize that such equivalent structures do not depart from the spirit and scope of the present invention, and various changes, substitutions and modifications can be produced without departing from the spirit and scope of the present invention.

1. A physiological sensing apparatus that is detachably arranged on a textile, the physiological sensing apparatus comprising:
a sensing module that receives a contact event to generate a physiological sensing signal, the sensing module comprising:
a sensor element that detects the physiological sensing signal; and
a first film having a first surface that comprises a conductive ink pattern, wherein a first surface of the sensor element is superimposed on the first surface of the first film such that the conductive ink pattern is in contact with the sensor element; and
a processing module coupled to the sensing module, wherein the processing module receives the physiological sensing signal to determine a physiological event corresponding to the physiological sensing signal.

2. The physiological sensing apparatus according to claim 1, wherein the sensing module further comprises a conductive film that covers an electrode pattern of the conductive ink pattern such that the conductive film is between the electrode pattern and the sensor element.

3. The physiological sensing apparatus according to claim 1, wherein the sensor element further comprises a second film arranged to cover the sensor element and a circuit pattern of the conductive ink pattern.

4. (canceled)

5. The physiological sensing apparatus according to claim 1, wherein the conductive ink pattern further comprises a comb structure, and wherein the comb structure comprises a plurality of strip-shaped resistors arranged in parallel, and first ends of the resistors in a same direction are connected to each other such that the resistors form a parallel circuit.

6. The physiological sensing apparatus according to claim 1, wherein the conductive ink pattern further comprises a comb structure, wherein the comb structure comprises a plurality of strip-shaped first resistors arranged in parallel and a plurality of strip-shaped second resistors arranged in parallel, wherein first ends of the plurality of first resistors are connected to each other such that the plurality of first resistors form a parallel circuit, and second ends of the plurality of second resistors are connected to each other such that the plurality of second resistors form a parallel circuit, wherein the first ends are opposite to the second ends.

7. The physiological sensing apparatus according to claim 6, wherein the plurality of first resistors and the plurality of second resistors are arranged alternately with each other.

8. The physiological sensing apparatus according to claim 3, wherein the sensing module further comprises a spacer arranged on a second surface of the sensor element and a first surface of the second film, such that the spacer is between the sensor element and the second film, wherein the second surface of the sensor element is an opposite surface of the first surface of the sensor element.

9. The physiological sensing apparatus according to claim 1, wherein the sensor element is a fabric sensor, and wherein the fabric sensor comprises a protruding fabric portion structure.

10. (canceled)

11. (canceled)

12. The physiological sensing apparatus according to claim 1, wherein the conductive ink pattern comprises an electrode pattern and a circuit pattern.

13. The physiological sensing apparatus according to claim 1, wherein the sensor element comprises a semiconductor sensor or a semiconductor sensing module, wherein the semiconductor sensor comprises at least one of a temperature sensor and a pressure sensor, and the semiconductor sensing module comprises at least one of a Bluetooth module and a wireless transmission module.

14. (canceled)

15. The physiological sensing apparatus according to claim 1, wherein the first film comprises an elastic waterproof film.

16. The physiological sensing apparatus according to claim 1, wherein the sensing module comprises a conductive film printed with an elastic conductive ink.

17. The physiological sensing apparatus according to claim 1, further comprising a waterproof film, wherein the waterproof film is arranged above the sensor element and the first film such that the sensor element and the first film are sealed by the waterproof film.

18. (canceled)

19. The physiological sensing apparatus according to claim 1 wherein the processing module is fixed to a fabric wear or separated from the fabric wear.

20. The physiological sensing apparatus according to claim 1, wherein the sensing module comprises a first conductive fiber sensing portion arranged in a first sensing region of the textile and generates a first sensing signal of the physiological sensing signal, wherein the processing module further determines whether the physiological event is an erection event according to a change in the first sensing signal detected in the first sensing region by the first conductive fiber sensing portion.

21. The physiological sensing apparatus according to claim 1, wherein he sensing module further comprises a second conductive fiber sensing portion arranged in a second sensing region of the textile and coupled to the processing module, wherein the second conductive fiber sensing portion generates a second sensing signal of the physiological sensing signal, and the processing module further determines whether the physiological event is a sleep event according to a change in the second sensing signal detected in the second sensing region by the second conductive fiber sensing portion.

22. The physiological sensing apparatus according to claim 1, further comprising a gravity sensor coupled to the processing module and generates a three-axis signal, wherein, when the processing module determines that the physiological event is a sleep event, the processing module further determines whether the three-axis signal is received and determine a sleep posture state of the sleep event according to the three-axis signal, wherein the sleep posture state comprises a back lying state, a left lying state, a right lying state, a front lying state, a bed leaving state and a walking state.

23. The physiological sensing apparatus according to claim 1, wherein the sensing module further comprises a third conductive fiber sensing portion arranged in a third sensing region of the textile and coupled to the processing module, wherein the third conductive fiber sensing portion generates a third sensing signal of the physiological sensing signal for the processing module to determine at least one of a heart rhythm state, a breathing state and a body temperature state of the physiological event.

24. The physiological sensing apparatus according to claim 23, wherein the processing module further determines the heart rhythm state of the physiological event according to a change in the third sensing signal; and when determining that the change in the third sensing signal is abnormal, record the heart rhythm state as an abnormal state.

25. The physiological sensing apparatus according to claim 23, wherein the processing module further determines the breathing state of the physiological event according to a change in the third sensing signal; and when determining that the change in the third sensing signal is abnormal, record the breathing state as an abnormal state.

26. The physiological sensing apparatus according to claim 23, wherein the processing module further determines the body temperature state of the physiological event according to a change in the third sensing signal; and when determining that the change in the third sensing signal is abnormal, record the body temperature state as an abnormal state.

27. The physiological sensing apparatus according to claim 1, further comprising a storage medium coupled to the processing module, wherein the storage medium stores the physiological event associated with the physiological signal for the processing module to determine a current physiological event according to a change in the physiological signal.

28. The physiological sensing apparatus according to claim 1, further comprising a wireless transmission module coupled to the processing module, wherein the wireless transmission module transmits the physiological event to an electronic device, such that the electronic device displays information about the physiological event.

29. The physiological sensing apparatus according to claim 1, further comprising a battery module and a charging module, wherein the battery module is coupled to the processing module and the charging module, and the charging module charges the battery module, such that the battery module provides power to the processing module.

30. A physiological sensing method, applicable to a physiological sensing apparatus, wherein the physiological sensing apparatus comprises a sensing module and a processing module, wherein the sensing module comprises a sensor element and a first film having a first surface that comprises a conductive ink pattern, wherein a first surface of the sensor element is superimposed on the first surface of the first film, such that the conductive ink pattern is in contact with the sensor element, and wherein the physiological sensing method comprises:
generating, by the sensing module arranged on a textile, a physiological sensing signal;
transmitting the physiological sensing signal to the processing module; and
determining, by the processing module analyzing the physiological sensing signal, a physiological event corresponding to the physiological sensing signal.

31. The physiological sensing method according to claim 30, characterized in that a first conductive fiber portion of the sensing module is arranged in a first sensing region of the textile, wherein the physiological sensing method further comprises:
generating, by the first conductive fiber portion, a first sensing signal of the physiological sensing signal; and
determining, according to a change in the first sensing signal, whether the physiological event is an erection event.

32. The physiological sensing method according to claim 30, wherein a second conductive fiber portion of the sensing module is arranged in a second sensing region of the textile, wherein the physiological sensing method further comprises:
generating, by the second conductive fiber portion, a second sensing signal of the physiological sensing signal; and
determining, according to a change in the second sensing signal, whether the physiological event is a sleep event.

33-38. (canceled)

39. A physiological information service system, comprising:
a physiological sensing apparatus detachably arranged on a textile, wherein the physiological sensing apparatus comprises:
a sensing module that generates a physiological sensing signal, the sensing module comprising:
a sensor element that detects the physiological sensing signal, and
a first film having a first surface that comprises a conductive ink pattern, wherein a first surface of the sensor element is superimposed on the first surface of the first film such that the conductive ink pattern is in contact with the sensor element; and
a processing module coupled to the sensing module, wherein the processing module receives the physiological sensing signal to determine a physiological event corresponding to a change in the physiological sensing signal; and
an electronic device that establishes a network connection with the physiological sensing apparatus through a gateway;
wherein the electronic device receives the physiological event through the network connection to prompt a detection warning message according to the physiological event.

40. (canceled)
